(19) United States
(12) Patent Application Publication
(10) Pub. No.: US 2014/0276656 A1
(43) Pub. Date: Sep. 18, 2014

(54) SURGICAL DRAINAGE TUBE ASSEMBLY WITH CONCENTRIC TUBULAR MEMBERS

(71) Applicants: Xiaoming Bian, Dalian (CN); Frank Zheng, Kirkland, WA (US)

(72) Inventors: Xiaoming Bian, Dalian (CN); Frank Zheng, Kirkland, WA (US)

(21) Appl. No.: 13/836,622

(22) Filed: Mar. 15, 2013

(51) Int. Cl. A61M 27/00

(52) U.S. Cl. CPC A61M 27/00
USPC 604/541

(57) ABSTRACT

A drainage tube assembly comprises an inner tubular member and an outer tubular member. The inner tubular member has an inner wall, an outer wall, and a plurality of apertures extending from the outer wall thereof to the inner wall thereof. The outer tubular member has an inner wall, an outer wall, and a plurality of apertures extending from the outer wall thereof to the inner wall thereof. The inner tubular member and the outer tubular member are coaxial with the outer wall of the inner tubular member abutting the inner wall of the outer tubular member. Corresponding ones of the apertures of the inner tubular member and the apertures of the outer tubular member are aligned. The inner tubular member is slidably removable from the drainage tube assembly. The apertures of the inner and outer tubular members may be oblique relative a longitudinal axis of the drainage tube assembly.

### BACKGROUND OF THE INVENTION

1. Field of the Invention

The present invention relates to a surgical drainage device and, in particular, to a surgical drainage device with concentric tubular members.

2. Description of the Related Art

It is known to use drainage devices to drain fluid from a patient during pre-surgical procedures, surgical procedures, and post-surgical procedures. Conventional drainage devices comprise a drainage tube provided with a plurality of lateral apertures. A negative pressure is applied to the drainage tube which causes fluid to be drawn into the drainage tube and thereby drained from the patient. However, a common problem with conventional drainage devices is that the drainage tube may become clogged by blood clots formed in a lumen thereof. Coating the drainage tube with heparin and providing a sliding wire within the lumen of the drainage tube have both been proposed as means to prevent the formation of blood clots in the lumen of the drainage tube.

The use of drainage tubes with concentric tubular members has also been disclosed as a means of preventing clogging of the drainage tube. For example, U.S. Pat. No. 3,771,527 which issued on Nov. 13, 1973 to Ruisi discloses a drainage tube comprising inner and outer concentric tubular members. The tubular members each have lateral apertures adjacent their distal ends. There is a means for connecting the proximal end of the inner tubular member to a source of suction. A cap closes off the proximal end of the outer tube with the inner tube extending through the cap. It was found that applying a negative pressure or suction to the inner tubular member but not the outer tubular member minimized clogging of the inner tubular member.

### SUMMARY OF THE INVENTION

There is provided a drainage tube assembly comprising an inner tubular member and an outer tubular member. The inner tubular member has an inner wall, an outer wall, and a plurality of apertures extending from the outer wall thereof to the inner wall thereof. The outer tubular member has an inner wall, an outer wall, and a plurality of apertures extending from the outer wall thereof to the inner wall thereof. The inner tubular member and the outer tubular member are coaxial with the outer wall of the inner tubular member abutting the inner wall of the outer tubular member. Corresponding ones of the apertures of the inner tubular member and the apertures of the outer tubular member are aligned. The inner tubular member is slidably removable from the drainage tube assembly. The apertures of the inner tubular member and the apertures of the outer tubular member may be oblique relative a longitudinal axis of the drainage tube assembly.

There is also provided a drainage tube assembly comprising an inner tubular member, an intermediate tubular member, and an outer tubular member. The inner tubular member has an inner wall, an outer wall, and a plurality of apertures extending from the outer wall thereof to the inner wall thereof. The intermediate tubular assembly has an inner wall, an outer wall, and a plurality of apertures extending from the outer wall thereof to the inner wall thereof. The outer tubular member has an inner wall, an outer wall, and a plurality of apertures extending from the outer wall thereof to the inner wall thereof. The inner tubular member, the intermediate tubular member, and outer tubular member are coaxial. The outer wall of the inner tubular member abuts the inner wall of the intermediate tubular member. The outer wall of the intermediate tubular member abuts the inner wall of the outer tubular member. Corresponding ones of the apertures of the inner tubular member, the apertures of the intermediate tubular member, and the apertures of the outer tubular member are aligned. The inner tubular member and intermediate tubular member are slidably removable from the drainage tube assembly. The apertures of the inner tubular member, the apertures of the intermediate tubular member, and the apertures of the outer tubular member may be oblique relative a longitudinal axis of the drainage tube assembly.

### BRIEF DESCRIPTION OF THE DRAWINGS

The invention will be more readily understood from the following description of the embodiments thereof given, by way of example only, with reference to the accompanying drawings, in which:

FIG. 1 is a perspective view of an improved surgical drainage device;

FIG. 2 is a sectional view of a drainage tube assembly of the surgical drainage device of FIG. 1 taken along lines A-A;

FIG. 3 is a sectional view of the drainage tube assembly of the surgical drainage device of FIG. 1 taken along lines B-B;

FIG. 4 is a sectional view of the drainage tube assembly of the surgical drainage device of FIG. 1 showing an inner tubular member partially removed from the drainage tube assembly;

FIG. 5 is a sectional view of the drainage tube assembly of the surgical drainage device of FIG. 1 with the inner tubular member removed;

FIG. 6 is a sectional view of the drainage tube assembly of the surgical drainage device of FIG. 1 showing an intermediate tubular member partially removed from the drainage tube assembly;

FIG. 7 is a sectional view of the drainage tube assembly of the surgical drainage device of FIG. 1 with the intermediate tubular member removed;

FIG. 8 is a sectional view of another drainage tube assembly which may be part of an improved surgical drainage device; and

FIG. 9 is a sectional view of yet another drainage tube assembly which may be part of an improved surgical drainage device.

### DESCRIPTION OF THE PREFERRED EMBODIMENTS

Referring to the drawings and first to FIG. 1, an improved surgical drainage device 10 is shown. The drainage device 10 generally comprises a drainage tube assembly 12 and a suction source 14, shown in fragment, which applies a negative pressure or suction to the drainage tube. The drainage tube assembly 12 has a rounded distal end 16 and the suction source 14 is in fluid communication with a proximal end 18 of the drainage tube assembly 12. The drainage tube assembly 12 is shown in greater detail in FIG. 2 and comprises concentric tubular members 20, 22 and 24. The inner tubular member 20 has an inner wall 26 and an outer wall 28. Likewise, the intermediate tubular member 22 has inner wall 30 and an outer wall 32, and the outer tubular member 24 has an inner wall 34 and an outer wall 36. The outer wall 28 of the inner tubular member 20 abuts the inner wall 30 of the intermediate tubular member 22 and the outer wall 32 of the intermediate tubular member 22 abuts the inner wall 34 of the outer tubular member 22.

Each of the tubular members 20, 22 and 24 further has a plurality of lateral apertures, for example, lateral apertures 38 and 40 shown for the inner tubular member 20, lateral apertures 42 and 44 shown for the intermediate tubular member 22 and lateral apertures 46 and 48 shown for the outer tubular member 20. The lateral apertures are disposed near the distal end 16 of the drainage tube assembly 12 and, in this example, are oblique relative to a longitudinal axis 100 of the drainage tube assembly 12. The oblique apertures facilitate the discharge of blood clots. Corresponding ones of the lateral apertures in the tubular members are aligned when the inner tubular member 20 is fully inserted into the intermediate tubular member 22 and the intermediate tubular member is fully inserted into the outer tubular member 24 and the lateral apertures are rotationally aligned. This is shown in FIG. 3 for lateral apertures 38, 42 and 46. Accordingly, when a suction pressure is applied to a lumen 50 of the inner tubular member 20, fluid is drawn into the lumen 50 of the inner tubular member 20.

The inner tubular member 20 may be slidably from removed drainage tube assembly 12 as shown in FIG. 4. The inner tubular member 20 may be removed from the drainage tube assembly 12 when the lumen 50 of the inner tubular member 20-becomes clogged by, for example, blood dots. The suction source 14, shown in FIG. 1, then applies suction to a lumen 52 of the intermediate tubular member 22 which is shown in FIG. 5. The intermediate tubular member 22 may also be slidably removed from the drainage tube assembly 12 as shown in FIG. 6. The intermediate tubular member 22 may be removed from the drainage tube assembly 12 when the lumen 52 of the intermediate tubular member 22 becomes clogged again by, for example, blood clots. The suction source 14, shown in FIG. 1, then applies a negative pressure or suction to a lumen 54 of the outer tubular member 24 which is shown in FIG. 7. The surgical drainage device 10 disclosed herein thereby allows the drainage tube assembly 12 to be unclogged without removing the drainage tube assembly 12 from a patient since the outer tubular 24 may remain in the patient when the inner tubular member 20 and intermediate tubular member 22 are removed from the drainage tube assembly 12. This reduces the risk of infection and other complications.

FIGS. 8 and 9 show further drainage tube assemblies which may be part of the improved drainage device as disclosed herein. The drainage tube assembly 60 shown in FIG. 8 is substantially similar to the drainage tube assembly 12 shown in FIG. 2 with the notable exception that the drainage tube assembly 60 of FIG. 8 comprises only an inner tubular member 62 and an outer tubular member 64. The drainage tube assembly 70 shown in FIG. 9 is generally similar to the drainage tube assembly 12 shown in FIG. 2 with the notable exception that the drainage tube assembly 60 of FIG. 9 comprises an inner tubular member 72, two intermediate tubular members 74 and 76, and an outer tubular member 78.

It will be understood by a person skilled in the art that many of the details provided above are by way of example only, and are not intended to limit the scope of the invention which is to be determined with reference to the following claims.

What is claimed is:

1. A drainage tube assembly comprising:
an inner tubular member having an inner wall, an outer wall, and a plurality of apertures extending from the outer wall thereof to the inner wall thereof;
an outer tubular member having an inner wall, an outer wall, and a plurality of apertures extending from the outer wall thereof to the inner wall thereof; and
wherein the inner tubular member and the outer tubular member are coaxial, the outer wall of the inner tubular member abutting the inner wall of the outer tubular member and corresponding ones of the apertures of the inner tubular member and the apertures of the outer tubular member being aligned, the inner tubular member being slidably removable from the drainage tube assembly.

2. The drainage tube assembly as claimed in claim 1 wherein the apertures of the inner tubular member and the apertures of the outer tubular member are oblique relative a longitudinal axis of the drainage tube assembly.

3. A drainage tube assembly comprising:
an inner tubular member having an inner wall, an outer wall, and a plurality of apertures extending from the outer wall thereof to the inner wall thereof;
an intermediate tubular assembly having an inner wall, an outer wall, and a plurality of apertures extending from the outer wall thereof to the inner wall thereof;
an outer tubular member having an inner wall, an outer wall, and a plurality of apertures extending from the outer wall thereof to the inner wall thereof; and
wherein the inner tubular member, the intermediate tubular member and outer tubular member are coaxial, the outer wall of the inner tubular member abutting the inner wall of the intermediate tubular member and the outer wall of the intermediate tubular member abutting the inner wall of the outer tubular member, corresponding ones of the apertures of the inner tubular member, the apertures of the intermediate tubular member and the apertures of the outer tubular member being aligned, and the inner tubular member and intermediate tubular member being slidably removable from the drainage tube assembly.

4. The drainage tube assembly as claimed in claim 1 wherein the apertures of the inner tubular member, the apertures of the intermediate tubular member, and the apertures of the outer tubular member are oblique relative a longitudinal axis of the drainage tube assembly.
